(19) United States
(12) Patent Application Publication
(10) Pub. No.: US 2005/0051161 A1
(43) Pub. Date: Mar. 10, 2005

(54) Alerting inhaler for inhalation therapy

(76) Inventors: Anand Vishnu Anandampillai, Chennai (IN); Vijayan Thirumalai Anandampillai, Chennai (IN)

Correspondence Address: T A V Jayan, 19 1St Parthasarathy Nagar, Adambakkam Chennai, Tamilnadu, 600088, IN

(21) Appl. No.: 10/485,230

(22) PCT Filed: Dec. 24, 2001
(86) PCT No.: PCT/IN01/00224

(30) Foreign Application Priority Data
Aug. 10, 2001 (IN) 647/MAS/00

(51) Int. Cl. A61M 11/00; A62B 7/00

(52) U.S. Cl. 128/200.23; 128/205.23

(57) ABSTRACT

The alerting inhaler has a smaller air flow tube, with a front air inlet closed by a cover with a hole for slow, deep inhalation. An air inlet leads to alert giving whistle or vibrator or rotating vanes and longer mouth piece. The long spray rod of the drug vial with drug(s) in solvent, propellant, enters to air tube and ends in a novel jet spray nozzle at back of mouth piece. A handle for support, helps easy pressing of drug vial on the body. The long mouth piece is kept in mouth, air is sucked, alert is heard, drug vial is pressed, the spray is released at back of throat for easy flow into wind pipes and lungs for good relief. The inhaler gives an alert to time the spray, with delivery of spray at the back of throat and not outside mouth as now, has no mouth wastage, and reliably delivers drugs to lungs for good effect even for children and elderly persons!

### TECHNICAL FIELD

This invention in general relates to human necessities. More particularly this invention relates to medical equipments. Precisely this invention relates to a novel kind of inhaler for successfully delivering drugs to lungs where the drug is absorbed and works, producing relief.

### STATE OF ART

Existing Inhalers used by Asthmatics (kids to aged) are difficult to coordinate in stress leading to poor relief. An alerting inhaler helps timing of spray delivery into lungs correctly. The following description gives critical examination of the inhalers known in the art. It also discusses the shortcomings of the conventional inhaler. Further in order to over come the problem associated with prior art inhalers, the invention offers the solution to overcome the impediments in the construction and the process of using the inhaler.

Constraints in the conventional device are as follows:
1. No alert mechanism
2. Spray starts outside the lip and spreads wasting spray into mouth.
3. Larger Air Inlet which produces shallow, fast inhalation and allows little time for coordination.
4. Large Mouth Piece which is difficult for children and elderly to hold in mouth
5. Mouth Wastage due to combination of above factors.

Accordingly the object of the invention is to identify the problem associated with the conventional inhalers. An extensive search has been carried out using the internet and related patent specifications were studied. Since the present invention is radically different, the inventor is unable to site any patent specification out of the available databases.

The invention is directed to a novel type of inhaler which is most effective for children and aged who may not be in a position to coordinate the spray triggering leading to poor utilization of the inhaler.

Further the invention is addressed to the process of using the new inhaler.

Accordingly it is the primary object of the invention to invent a novel inhaler which is unique in design and construction, working and use.

It is another object of the invention to indicate a novel process by virtue of it. The inhaler can be effectively utilized.

It is another object of this invention is to invent a novel inhaler which has an alert mechanism which helps to trigger the device correctly.

It is another object of this invention is to invent a novel inhaler which allows slow and deep inspiration of air, which gives more time for spray release and spread of spray deeply into the lungs.

Further objects of the invention will be clear from the ensuing description.

### SUMMARY OF THE INVENTION

An inhaler comprising of a smaller air inlet guarded by a stopper with an appropriate hole, the said air inlet leads to a whistle (or vibrator or rotating wheel), the said whistle leads to a spray rod with jet spray nozzle, the said spray path leads to a mouth piece for the spray with the inspired air to flow into the lungs. The drug vial with the desired drugs in solvent and propellants is fixed in a body incorporated with the air path for easy pressing and release of spray.

### STATEMENT OF DRAWINGS

These and other objects and features of the invention will become more apparent upon perusal of following description taken in conjunction with accompanying drawings wherein:

FIG. 1 shows in elevation the constructional details of conventional inhaler.

FIG. 2 shows the various components of the novel inhaler according to invention.

FIG. 3 shows the exploded view of the novel inhaler.

FIG. 4 shows the mechanism and spread of the drug mist originating from the novel inhaler inside the mouth.

FIG. 5 shows another embodiment of the invention.

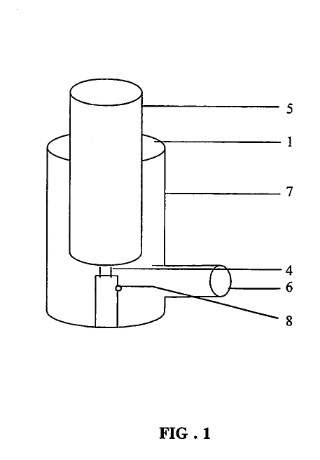

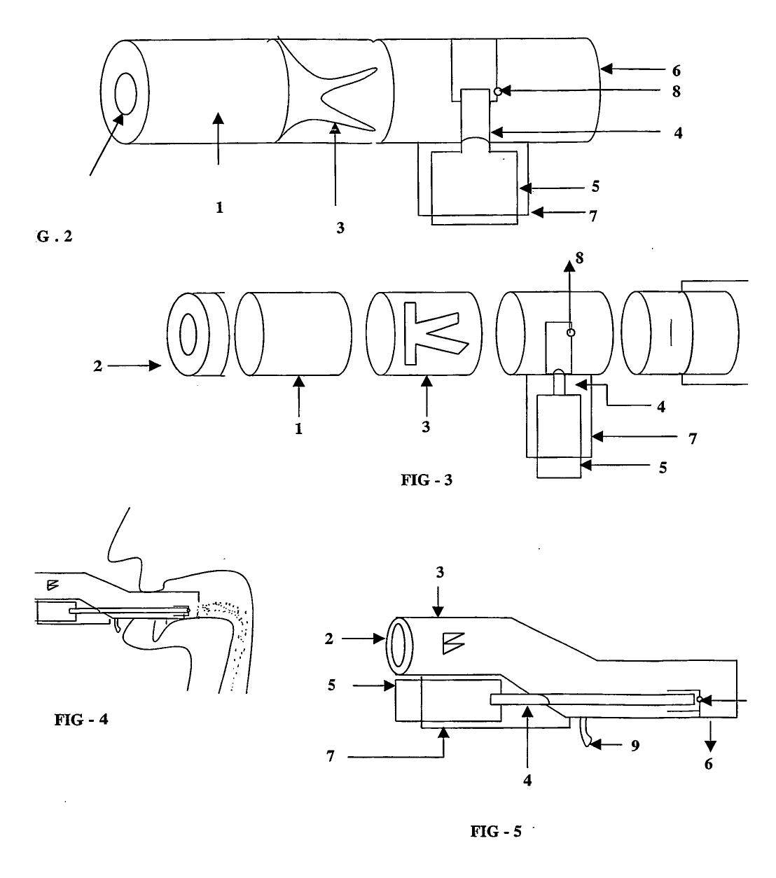

### DESCRIPTION OF PREFERRED EMBODIMENTS

The following specification describes salient features of invention, the method of construction, the method of use and the advantages of the novel invention.

The novel inhaler has smaller air inlet with a cover, with a hole to develop deep, longer inspiration. A whistle, behind the air inlet gives sound alert for spray trigger. The whistle leads to the spray rod of a drug vial, with a fine hole to back spray into an adjustable mouthpiece. The mouthpiece directs the spray into wind passages and lungs for good effect.

The novel inhaler according to the invention is kept in mouth, air is sucked in through the air inlet, the whistle gives an alert, the drug vial is now pressed to release a spray at the back of mouth piece which travels to lungs for better effect.

The novel inhaler according to invention is better because of alert, slow, deeper inspiration, adjustable mouth piece and air inlet for an individual variation.

Special feature of the invention is that it is an alerting inhaler with improved spray useful for inhalation therapy. Existing inhalers spray the mouth, have no alert (difficult for kids and aged) and do not facilitate deep inspiration for drug spread in the lungs.

The novel inhaler according to invention gives a whistle or vibration to alert the user to spray (now made easy for kids & aged), directs more spray into wind passages and promotes longer and deeper inspiration for the spray to spread to all areas of the lungs.

One of the salient feature of the invention is that the novel inhaler according to invention has a narrower air flow pipe comprising of a smaller air inlet with a hole (adjustable for slow deep inspiration) a whistle as the air is sucked, a spray rod with a fine hole and a back mouth piece (also adjustable now, for lips to throat distance). The same volume of air sucked through a smaller area takes longer time. The chest muscles also work deeper for forceful inhalation and spread of drugs. The sound also decreases the tension.

The whistle alerts for correct spray before maximal inspiration, the smaller air inlet delays and deepens inspiration, and the mouthpiece is adjustable for correct spray into throat and wind pipes leading to lungs.

The conventional inhaler consists of a body (7) with a large air inlet (1) at the top in which the drug spray vial (5) is placed. The body has at its other end has a large area mouth piece (6). The body on its inside has a jet spray nozzle (8) which receives the spray rod (4), the drug vial (5). To use, the mouth piece (6) is kept in mouth. Air is inhaled. Inhalation is shallow and fast as the area is large. There is no time to trigger. There is no alert. There is wastage of spray in the mouth as the spray is released outside the lip level. Children and elderly persons do not coordinate the spray release. There is less response to the drug and device. The device has not been improved for decades. The object of the invention is to overcome the inherent defect in the device. To this end the new invention is addressed.

The new inhaler comprises of an air suction tube preferably of plastic or metal. At the front is the air inlet (1), with a cover (2) with the device is a new inhaler. The air inlet leads to a whistle or vibrator (3) or rotating vane which helps to alert and time the spray in inspiration correctly and followed by a spray rod (4) of the drug spray vial (5). The spray rod fits the jet nozzle (8) to spray the drug back into wind passages. The mouthpiece (6) is preferably formed of two concentric tubes, the outer sliding on the inner by a screw thread or sliding slot for adjustment of lips to throat distance, releasing the spray at the back of throat! The drug vial is inserted in the body (7) of the inhaler. Note that air is not drawn around the drug vial but through the small separate inlet air vial.

The novel device alerts the user to trigger the device in inspiration, does not spray into mouth, novely directs flow of mist maximally to air passages delivering correct doses, and is safe for children, aged, even in disorientation!

The novel device's mouthpiece is kept between the lips. The air inlet orifice allows air to be sucked in inspiration! The orifice may be adjusted for deep and slow air suction! The suction of air rotates a small vane or a whistle or vibrator which alerts the user to press the drug vial! The whistle or vibrator or wheel is a novel indicator of suction of air in inspiration! The drug vial has the drug(s) in solvent with propellant to spray. The spray rod is longer and projects into the mouth longer as in FIG. 5. The jet nozzle produces a mist directed to the wind pipe and not to mouth! The drug is carried to distal air passages uniformly, because of deep and slow inhalation. The increased duration of inspiration also helps in spread of mist. The drug is delivered better maximally, without waste, with a visible or audible alarm for the user for the first time in a simple manner!

The inhaler is made of plastic with the orifices, whistle, nozzle incorporated as a unit or as separate segments easily assembled.

To use, the mouth piece is kept in the mouth after adjustment of lip-throat distance and inspiration started, which gives a whistle. The drug vial is pressed to release the spray into wind pipes and lungs. The deeper and prolonged inspiration spreads the drug spray to all parts of lungs, for a better effect.

In FIG. 5, another form of invention shows an air inlet (1) is on the upper side, with alert device (3) a whistle. The tube bends and straightens to form the long mouth piece (6). The drug vial (5) fits in the body (7), has now a longer spray rod (4), which enters at the bend out and ends in the jet nozzle (8) at the back of mouth piece (6). The air flow is not around the drug vial but by to separate smaller air inlet! A handle (9) steadies the inhaler on the thumb, other fingers press the drug vial for spray release! This is the ideal inhaler for adults with long lip to throat distance! A sealing washer allowing movement of the spray rod in the hole at the bend may be used. A filter to remove air bacteria may be used before the spray rod.

The device can be modified. The stopper of air inlet can have an adjustable hole or different stoppers with different holes may be used as the person wants for prolonged deep inspiration! The drug vial may be mounted as in FIG. 5 in the long axis of the inhaler! The mouth piece tubes may be adjusted on a screw or sliding mechanism. Electronic sensing and spraying is possible but will make the device costly and heavy. Whistle may produce sound of any type. For deaf people a vibrator of swaying metallic leaflets may be used. The flaps should be fixed as not to fly off. A rotating wheel in a transparent air inlet will also serve as alert to people who do not want sound alert but need visual clue.

The term drug vial means a unit comprising a can, a crimped cap covering the mouth of the can, and a drug metering valve situated in the cap, also includes a suitable hannelling device, which delivers a predetermined amount of drug formulation upon each ctivation. The channelling device may comprise, for example, an actuating device for the alve and a cylindrical or cone-like passage through which medicament may be delivered rom the filled can via the valve to the mouth of a patient, e.g. a mouthpiece actuator.

The term “drug formulation” means active drug (or a physiologically acceptable olvate thereof) optionally in combination with one or more other pharmacologically active gents such as other antiinflammatory agents, analgesic agents or other respiratory drugs and optionally containing one or more excipients, and a fluorocarbon propellant. The term “excipients” as used herein means chemical agents having little or no pharmacological activity (for the quantities used) but which enhance the drug formulation or the performance of the system. For example, excipients include but are not limited to surfactants, preservatives, flavorings, antioxidants, antiaggregating agents, and cosolvents, e.g., ethanol and diethyl ether.

A polar cosolvent such as C.sub.2-6 aliphatic alcohols and polyols e.g. ethanol, isopropanol and propylene glycol, preferably ethanol, may be included in the drug formulation in the desired amount, either as the only excipient or in addition to other excipients such as surfactants. Suitably, the drug formulation may contain 0.01 to 5% w/w based on the propellant of a polar cosolvent e.g. ethanol, preferably 0.1 to 5% w/w e.g. about 0.1 to 1% w/w.

It will be appreciated by those skilled in the art that the drug formulation for use in the invention may, if desired, contain one or more other pharmacologically active agents. Such medicaments may be selected from any suitable drug useful in inhalation therapy. Appropriate medicaments may thus be selected from, for example, analgesics, e.g. codeine, dihydromorphine, ergotamine, fentanyl or morphine; anginal preparations, e.g. diltiazem; antiallergics, e.g. cromoglycate, ketotifen or nedocromil; antiinfectives e.g. cephalosporins, pentamidine; antihistamines, e.g. methapyrilene; anti-inflammatories, e.g. beclomethasone, fluticasone propionate, flunisolide, budesonide, tipredane or triamcinolone acetonide; antitussives, e.g. noscapine; bronchodilators, e.g. salbutamol, salmeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, orciprenaline, or (−)-4-amino-3,5-dichloro-.alpha.[[[6-[2-(2-pyridinyl)ethoxy]hexyl]amino]methyl]benzenemethanol; diuretics, e.g. amiloride; anticholinergics e.g. ipratropium, atropine or oxitropium; hormones, e.g. cortisone, hydrocortisone or prednisolone; xanthines e.g. aminophylline, choline theophyllinate, lysine theophyllinate or theophylline; and therapeutic proteins and peptides, e.g. insulin or glucagon and genetic fragments. It will be clear to a person skilled in the art that, where appropriate, the medicaments may be used in the form of salts (e.g. as alkali metal or amine salts or as acid addition salts) or as esters (e.g. lower alkyl esters) or as solvates (e.g. hydrates) to optimise the activity and/or stability of the medicament and/or to minimize the solubility of the medicament in the propellant.

Particularly preferred drug formulations for Asthma contain fluticasone propionate (or a physiologically acceptable solvate thereof) in combination with a bronchodilator such as salbutamol (e.g. as the free base or the sulphate salt) or salmeterol (e.g. as the xinafoate salt). Combinations for the other diseases may be used for treatment.

“Propellants” used herein mean pharmacologically inert liquids with boiling points from about room temperature (25. degree. C.) to about −25. degree. C. which singly or in combination exert a high vapor pressure at room temperature. Upon activation of the drug vial, the high vapor pressure of the propellant in the MDI forces a metered amount of drug formulation out through the metering valve. Then the propellant very rapidly vaporizes dispersing the drug particles. The propellants used in the present invention are low boiling fluorocarbons; “propellant 227” or “P 227”, “propellant 134a”.

Drug formulations for use in the invention may be free or substantially free of formulation excipients e.g. surfactants and cosolvents etc. Such drug formulations are advantageous since they may be substantially taste and odour free, less irritant and less toxic than excipient-containing formulations. Thus, a preferred drug formulation consists essentially of fluticasone propionate, or a physiologically acceptable salt thereof, optionally in combination with one or more other pharmacologically active agents particularly salmeterol (e.g. in the form of the xinafoate salt), and a fluorocarbon propellant.

Further drug formulations for use in the invention may be free or substantially free of surfactant.

Most often the drug vial can and cap are made of aluminum or an alloy of aluminum, although other metals not affected by the drug formulation, such as stainless steel, an alloy of copper or tin plate, glass or plastic may be used.

The drug-metering valve consists of parts usually made of stainless steel, a pharmacologically inert and propellant resistant polymer, such as acetal, polyamide (e.g., Nylon.RTM.), polycarbonate, polyester, fluorocarbon polymer (e.g., Teflon.RTM.) or a combination of these materials. Additionally, seals and “O” rings of various materials (e.g., nitrile rubbers, polyurethane, acetyl resin, fluorocarbon polymers), or other elastomeric materials are employed in and around the valve.

Particularly preferred coatings for inside of drug vial are pure PFA, FEP and blends of PTFE and polyethersulphone (PES).

The particle size of the particular (e.g., micronised) drug should be such as to permit inhalation of substantially all the drug into the lungs upon administration of the aerosol formulation and will thus be less than 100 microns, desirably less than 20 microns, and, in particular, in the range of 1-10 microns, e.g., 1-5 microns.

The final drug formulation desirably contains 0.005-10% weight to weight ratio, in particular 0.005-5% weight to weight ratio, especially 0.01-1.0% weight to weight ratio, of drug relative to the total weight of the formulation.

The drug vial with fluorocarbon polymer/polymer blend coated interiors taught herein may be used in medical practice in a similar manner as non-coated MDI's now in clinical use.

It will be apparent to those skilled in the art that modifications to the invention described herein can readily be made without departing from the spirit of the invention. Protection is sought for all the subject matter described herein including any such modifications.

### Advantages of the New Invention

1. Slow and deep inhalation, as air is drawn through a smaller area of air inlet.

2. The device gives more time to trigger the spray.

3. There is an alert mechanism which is embodied for the first time to help time the trigger.

4. There is no need for washing the mouth after the device is used as there is no mouth spray.

5. Useful for kids, old, who can now coordinate the spray mechanism easily.

6. The spray is released at the back of the mouth and not outside the lip level, which easily goes into windpipes and lungs for good effect.

1) an alerting inhaler comprising of an air inlet guarded by a stopper with an appropriate hole to develop deep and longer inspiration, the said air inlet fixed to a whistle or vibrator, the said whistle gives sound alert for the drug spray trigger, the said whistle is fixed to a drug spray path, formed by spray rod of the drug vial with a fine jet nozzle to back spray into the adjustable mouth piece, the said mouth piece directs the spray into wind passages, such that the said drug spray with the inspired air flows into the lungs.

2) an alerting inhaler as claimed in claim 1, wherein the said air inlet has a cover at the front, the said cover having a hole preferably adjustable for entry of air.

3) an alerting inhaler as claimed in claim 1, wherein the said whistle or vibrator is placed before the spray, the said whistle is activated by the inspired air, the said whistle alerts to time the spray.

4) an alerting inhaler as claimed in claim 1, wherein the said spray path comprises of a drug spray vial with a spray rod, the said spray rod fixed behind the said whistle, the said rod fitting a jet nozzle for the spray to be formed, the said spray is directed back through the mouthpiece into lungs.

5) an alerting inhaler as claimed in claim 1, wherein the said mouthpiece comprises of an outer tubes sliding on the inner tube on a screw or slot to adjust lip to throat distance for correct spray.

6) an alerting inhaler as claimed in claim 1, wherein the said drug vial is placed along the long axis to the said air path.

7) an alerting inhaler as claimed in claim 1, wherein the said drug vial is placed at perpendicular to the air path.

8) an alerting inhaler as claimed in claim 1, wherein the vibrator is placed instead of the whistle before the said spray to alert the user by vibrations.

9) an alerting inhaler as claimed in claim 1, wherein a coloured rotating wheel is placed in a transparent enclosure is placed instead of whistle before the said spray to alert the user by visual clue.

10) an alerting inhaler as claimed in claim 1, wherein the inhaler is made of plastic or metal.

11) an alerting inhaler as claimed in claim 1, wherein the drug vial comprises of strong metal can with a, crimped cap covering the mouth of the can and a drug metering valve in the cap.

12) an alerting inhaler as claimed in claim 1, wherein the drug formulation comprises of an active agent for the medical need, as a micronised particle, in a solvent, with a propellant with or without surfactants or exceipients the said drug when sprayed reaches the lung for rapid relief.

13) an alerting inhaler as claimed in claim 1, wherein a handle is fixed to the body to steady and easily press the drug vial.

14) an alerting inhaler as claimed in claim 1, wherein the inhalation drug may be single or combination of active pharmacological agents as described in the specification.

15) A method of spraying the contents of drug vial for inhalation therapy in the mouth comprising of
(a) Placing the inhaler jet spray at the depth of mouth so that spray is released at the back of the mouth,
(b) Use an alert mechanism for effective spray using alert trigger which gives more effective spray in correct path of inhalation.
(c) Effecting slow and deep inhalation of air through a smaller hole of inhaler air inlet such that the inspired air flows into lungs with drugs for relief.

16) An alerting alerting inhaler for inhalation therapy as described in the specification and as illustrated by way of drawings.

17) A method of spraying the contents of vial in the mouth as described in the specification.
